(19) United States
(12) Patent Application Publication
(10) Pub. No.: US 2018/0051132 A1
(43) Pub. Date: Feb. 22, 2018

(54) ELASTOMER COUPLED WITH BIOACTIVE FATTY ACID

(71) Applicant: University of Pittsburgh - Of the Commonwealth System of Higher Education, Pittsburgh, PA (US)

(72) Inventors: Yadong Wang, Bradfordwoods, PA (US); Soo Hyon Lee, Pittsburgh, PA (US)

(73) Assignee: University of Pittsburgh - Of the Commonwealth System of Higher Education, Pittsburgh, PA (US)

(21) Appl. No.: 15/681,152

(22) Filed: Aug. 18, 2017

Related U.S. Application Data
(60) Provisional application No. 62/376,764, filed on Aug. 18, 2016.

(51) Int. Cl. C08G 63/48

(52) U.S. Cl. CPC C08G 63/48; A61F 2/06

(57) ABSTRACT

A polymer comprising repeating units having a structure of:

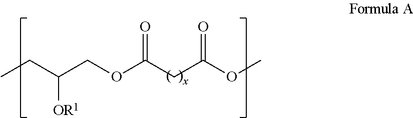

wherein x is 2 to 25; and
each R¹ is the same or different and is a moiety derived from an unsaturated fatty acid, a dicarboxylic acid, or a dicarboxylic acid-terminated polymer.

This application claims the benefit of U.S. Provisional Appl. No. 62/376,764, filed Aug. 18, 2016, which is incorporated herein by reference in its entirety.

### BACKGROUND

Biodegradable synthetic polymers have been widely explored for their great potential in soft tissue engineering. In particular, over the last two decades, members of the polyester family such as polyglycolide or polylactide have been developed to be used as scaffolds for tissue regeneration due to their biomimetic mechanical properties and its tunable biodegradability.

### SUMMARY

Disclosed herein is a polymer comprising repeating units having a structure of:

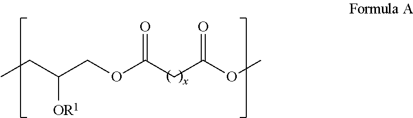

wherein x is 2 to 25; and

each R¹ is the same or different and is a moiety derived from an unsaturated fatty acid, a dicarboxylic acid, or a dicarboxylic acid-terminated polymer.

Also disclosed herein is a polymer comprising an unsaturated fatty acid-functionalized poly(glycerol diacid).

Further disclosed herein is a prepolymer comprising:

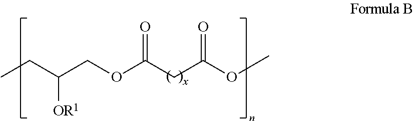

wherein n is 2 to 12;

x is 2 to 25; and

each R¹ is the same or different and is a moiety derived from an unsaturated fatty acid, a dicarboxylic acid, or a dicarboxylic acid-terminated polymer.

Additionally disclosed herein is a porous scaffold comprising a polymer disclosed herein.

Also disclosed herein is a vascular graft comprising a polymer disclosed herein.

Further disclosed herein are methods comprising reacting together a poly(glycerol diacid) and an unsaturated fatty acid.

The foregoing will become more apparent from the following detailed description, which proceeds with reference to the accompanying figures.

### BRIEF DESCRIPTION OF THE DRAWINGS

FIG. 1 is a graph showing mean contact angle data for inventive poly(glycerol sebacate linoleate) (PGSL) and comparative poly(glycerol sebacate) (PGS).

FIG. 2 is a DSC graph.

FIG. 3 shows a cell viability live/dead assay. 5,000 HUVEC were incubated in extract media of PGS and PGSL at 37° C. for 24 hrs. By treating with calcein AM (live cell marker) and ethidium homodimer-1 (dead cell marker), fluorescent images of cells in the FITC (calcein, green) and TRITC (ethidium, red) channels of fluorescence microscopy.

FIG. 4 shows subcutaneous implantation of PGSL in mice. PGS completely degraded after 2 weeks, PGSL was maintained even after 4 weeks in vivo. The hydrophobicity and the structure of PGSL may decrease the degradation rate of the material. Histology staining with hematoxylin and eosin (H&E) shows aggregation of cells in the injured area.

FIG. 5 shows in vivo cell migration. CD68/DAPI staining—PGS and PGSL implants in mice at 3 different time points (Day 3, week 2, and week 4). Pronounced CD68 (macrophage and monocyte marker, TRITC) was observed in the injured area at Day 3. This result shows that a large amount of inflammatory cells (IC) and also other type of cells (stained by DAPI) in PGSL at week 2 and week 4.

FIG. 6 shows endothelial cell (EC) immunofluorescence after 4 weeks of implantation. CD31/DAPI staining—PGS total degraded at week 4. At week 4, no significant presence of EC was observed. In PGSL form, high CD31 positive cells were observed. Some of the EC in PGSL scaffold are already regrouping and forming blood vessel morphology.

FIG. 7 shows blood vessel formation after 2 weeks and 4 weeks of implantation of PGSL scaffold. CD31/α-SMA(α-smooth muscle cell actin)/DAPI co-staining permits to observe vascular EC and the mural cells of the blood vessels. We observed significant aggregation of ECs (red) and vascular smooth muscle cells (SMCs) (green).

FIGS. 8A-8C show sheep vascular endothelial cell (SVEC) proliferation in the presence of linoleic acid. 5000 SVEC were cultured in different concentration of LA 3.6 μM, 360 nM and 36 nM with and without nutrition and growth factors. SVEC incubated in 360 LA and 36 LA without growth factors shows higher proliferation than the cells in the positive control medium. 36 μM M LA was too acidic for medium preparation. pH of EGM-2 basal medium became too acidic to culture cells. (−) Plain EGM-2 basal medium, (+) basal medium with FBS fetal bovine serum, (++) basal medium with FBS and growth factors.

FIG. 9 is a graph showing in vitro degradation rate of PGS and PGSL.

FIG. 10 is a graph showing in vivo degradation rate of PGS and PGSL.

FIG. 11 is a graph showing vascular cell evolution in a PGSL scaffold.

FIG. 12 is a graph showing endothelial cell quantity variation.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

Anticoagulant: A substance that prevents the clotting of blood (coagulation). Anticoagulants are commonly administered to subjects to prevent or treat thrombosis. Generally, anticoagulants are administered to treat or prevent deep vein thrombosis, pulmonary embolism, myocardial infarction, stroke, and mechanical prosthetic heart valves. Various types of anticoagulants with different mechanisms of action are available including anticoagulants that inhibit the effect of vitamin K (such as coumadin) or thrombin directly (such as argatroban, lepirudin, bivalirudin, and ximelagatran) or that activate antithrombin II that in turn blocks thrombin from clotting blood (such as heparin and derivative substances thereof).

Bioactive agents: As used herein, “bioactive agents” is used to refer to compounds or entities that alter, inhibit, activate, or otherwise affect biological or chemical events.

Biocompatible: A term describing something that can be substantially non-toxic in the in vivo environment of its intended use, and is not substantially rejected by the patient's physiological system (e.g., is nonantigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of subjects, will not cause a significantly adverse reaction or response. Furthermore, biocompatibility can be affected by other contaminants such as prions, surfactants, oligonucleotides, and other agents or contaminants. The term “biocompatible material” refers to a material that does not cause toxic or injurious effects on a tissue, organ, or graft.

Biodegradable polymer: A polymer that can be cleaved either enzymatically or hydrolytically to break it down sufficiently so as to allow the body to absorb or clear it away. In certain embodiments, a biodegradable polymer is a polymer that degrades fully (i.e., down to monomeric species) under physiological or endosomal conditions. A biodegradable vascular graft is a graft in which at least a significant portion (such as at least 50%) of the graft degrades within one year of implantation.

Physiological conditions: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

Poly(glycerol sebacate) (PGS): An elastomeric biodegradable polyester. In some examples, PGS is electrospun with gelatin to form fibrous constructs. In some examples, PGS prepolymer is blended with a synthetic polymer such as polyvinyl alcohol (PVA), polyhydroxybuytrate (PHB) or polyethylene terephthalate (PET). In some examples, PGS prepolymer is blended with poly(lactic-co-glycolic acid) (PLGA) and a chemical cross-linker, then electrospun as a blended material. The PLGA is removed with organic solvent to leave only PGS fibers. In some examples, PGS prepolymer is blended with gelatin. In some examples, PGS is electrospun, a sacrificial template is placed on a sheet of electrospun PGS and a second layer of PGS fibers are electrospun on top of the sacrificial template.

Scaffold: A structural support facilitating cell infiltration and attachment in order to guide vessel growth. As disclosed herein, a biodegradable scaffold can be used to form a vascular graft. In some examples, a biodegradable scaffold includes PGSL.

Subject: Living multi-cellular vertebrate organisms, a category that includes human and non-human mammals (such as laboratory or veterinary subjects). In an example, a subject is a human. In an additional example, a subject is selected that is in need of an implant for damaged or defective artery.

Vascular graft: A tubular member which acts as an artificial vessel. A vascular graft can include a single material, a blend of materials, a weave, a laminate or a composite of two or more materials.

### Material and Methods of Making

Disclosed herein is a novel biodegradable polymer which in certain embodiments may promote enhancement of angiogenesis to achieve efficient in situ tissue regeneration for vascular disease. In one embodiment the polymer comprises an unsaturated fatty acid-functionalized poly(glycerol diacid). For example, disclosed herein is a PGS-derived polymer that includes a pro-angiogenic fatty acid, (e.g., poly(glycerol sebacate linoleate) (PGSL)), and is synthesized by polycondensation of three biomolecules: glycerol; sebacic acid; and linoleic acid. Controlled release of angiogenic fatty acid by the biodegradability of the PGSL enhances in situ vascular tissue regeneration. The biocompatibility of PGSL was determined by in vitro cell viability assay and by subcutaneously implanting the PGSL into mice. Biodegradability tests were also carried out in physiological conditions in the presence of enzyme and in Dulbecco's phosphate buffered saline (DPBS). Vascular endothelial cell migration was monitored after implantation of PGSL in mice, through the release of the pro-angiogenic fatty acid units during degradation of the PGSL. The biodegradability and enhanced angiogenesis of PGSL together is a promising candidate for a new class of scaffold for blood vessel regeneration.

The enhancement of blood vessel formation has been improved by applying, for the first time, an angiogenesis enhancer that differs from conventional growth factors. A fatty acid that is nontoxic (e.g., linoleic acid) is covalently incorporated into a polymeric scaffold, and controlled release of the fatty acid was observed for enhancing angiogenesis in regeneration of soft tissue.

Certain embodiments of the PGSL may include the following features:
Linoleic acid is successfully incorporated into a PGS backbone by polycondensation;
Excellent biocompatibility of PGSL in vitro and in vivo assay;
A large amount of cells were infiltrated into the PGSL material during the degradation;
Consistent presence of inflammatory cells (IC) and endothelial cells (EC) during the degradation; and/or
Scaffold material is biocompatible and degradable.

In one embodiment the polymer comprises an unsaturated fatty acid-functionalized poly(glycerol diacid). Illustrative unsaturated fatty acids include a polyunsaturated omega-6 fatty acid, for example, linoleic acid (LA), gamma linolenic acid (GLA), arachidonic acid (AA), epoxyeicosatrienoic acid, or conjugated linoleic acid (CLA); or a polyunsaturated omega-3 fatty acid (e.g.,docohexaenoic acid). Illustrative diacids include sebacic acid, malonic acid [HOOC(CH₂)COOH], succinic acid [HOOC(CH₂)₂COOH] up to long chain fatty acid dimers, may also be used to form elastomeric biomaterials according to the invention. Exemplary diacids include glutaric acid (5 carbons), adipic acid (6 carbons), pimelic acid (7 carbons), suberic acid (8 carbons), and azelaic acid (nine carbons). Exemplary long chain diacids include diacids having more than 10, more than 15, more than 20, and more than 25 carbon atoms. In certain embodiments, the molar ratio of glycerol:diacid:unsaturated fatty acid is 1:1:1.

Also disclosed herein embodiment is a polymer comprising units having a structure of:

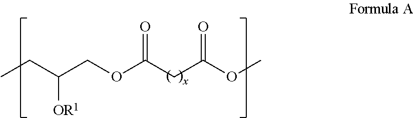

wherein x is 2 to 25; and

each R¹ is the same or different and is a moiety derived from an unsaturated fatty acid, a dicarboxylic acid, or a dicarboxylic acid-terminated polymer.

Further disclosed herein is a prepolymer comprising repeating units having a structure of:

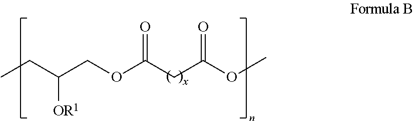

wherein n is 2 to 12;

x is 2 to 25; and

each R¹ is the same or different and is a moiety derived from an unsaturated fatty acid, a dicarboxylic acid, or a dicarboxylic acid-terminated polymer.

Each R¹ is the same or different and is a moiety derived from an unsaturated fatty acid, a dicarboxylic acid, or a dicarboxylic acid-terminated polymer. For example, R¹ may be derived from a polyunsaturated omega-6 fatty acid, for example, linoleic acid (LA), gamma linolenic acid (GLA), arachidonic acid (AA), epoxyeicosatrienoic acid, or conjugated linoleic acid (CLA). In another example, R¹ may be derived from a polyunsaturated omega-3 fatty acid (e.g.,docohexaenoic acid). In certain embodiments, at least one, and more particularly a plurality of, R¹ group(s) is linoleate, gamma linolenate, arachidonate, epoxyeicosatrienoate, conjugated linoleate, or docohexaenoate. In certain embodiments, at least one R¹ is linoleate and at least one R¹ is sebacate.

The prepolymer may be cured wherein the repeating units of formula A or formula B are crosslinked via attachment at the R¹ position. For example, a first unit of formula A or formula B may be crosslinked with a further unit of formula A or formula B via the R¹ position resulting in a crosslinked, polymeric network. In other words, in the crosslinked polymer a plurality of R¹ groups are dicarboxylic acid-terminated polymer chain derived from units of formula A or formula B.

In certain embodiments, the cured unsaturated fatty acid-functionalized poly(glycerol diacid) may have a Young's modulus of tensile elastic modulus of 0.174 to 0.202 MPa, more particularly 0.180 to 0.192 MPa.

In certain embodiments, the unsaturated fatty acid-functionalized poly(glycerol diacid) material may be prepared by curing a mixture of a poly(glycerol diacid) (e.g., PGS) prepolymer and the fatty acid. The poly(glycerol diacid) prepolymer may be prepared, for example, as described in U.S. Pat. No. 7,722,894, which is incorporated herein by reference. Preferred molar ratios for glycerol-diacid co-polymers may range from 1:1 to 1:1.5. Catalysts may be used to reduce reaction temperature, shorten reaction time, and increase individual chain length. However, the catalyst should be bio-compatible or easily removed. An exemplary FDA-approved catalyst is stannous octoate (bis(2-ethylhexanoate)tin(II)), available from Fluka and Strem.

The direct addition of the fatty acid at the curing step is simple, faster, and does not require an additional catalyst. The fatty acid may be an unsaturated fatty acid, particularly an unsaturated fatty monoacid such as a polyunsaturated omega-6 fatty acid or a polyunsaturated omega-3 fatty acid as described above. In certain embodiments, the fatty acid is a bioactive fatty acid that may provide a therapeutic benefit to a subject. For example, the fatty acid may be a pro-angiogenic agent and/or anti-inflammatory agent such as linoleic acid, arachidonic acid, or epoxyeicosatrienoic acid. In a further example, the fatty acid may an anti-angiogenesis agent such as gamma CLA or docosahexaenoic acid. Alternatively, the unsaturated fatty acid-functionalized poly(glycerol diacid) material may be prepared, for example, by fabricating a unsaturated fatty acid-functionalized poly(glycerol diacid) prepolymer and then curing to form the final crosslinked polymer.

For example, the PGSL may be synthesized by mixing together the fatty acid (e.g., linoleic acid) and a PGS prepolymer and then curing the mixture to form PGSL. The curing may be thermal curing (e.g., subjecting the mixture to 120 to 150° C., more particularly 120 to 140° C.), with or without vacuum. In certain embodiments, the fatty acid/PGS prepolymer mixture is initially subjected to 120° C. for 2 to 4 hours without vacuum, and then subjected to 140° C. for 20 to 24 hours under a vacuum of 20 to 100 mTorr.

The fatty acid forms a covalent bond via a hydroxyl group on the poly(glycerol diacid) prepolymer backbone. The addition of the fatty acid (e.g., linoleic acid) does not affect the elongation of repeat unit of the PGS prepolymer, as it is a monoacid which has only one functional end. Thus, the monoacid fatty acid can be considered as an end capping agent. In other words, the unsaturated fatty acid forms a pendant and/or terminal group on the poly(glycerol diacid) network backbone.

Diacids other than sebacic acid of different lengths, including malonic acid [HOOC(CH₂)COOH] and succinic acid [HOOC(CH₂)₂COOH] up to long chain fatty acid dimers, may also be used to form elastomeric biomaterials according to the invention. Exemplary diacids include glutaric acid (5 carbons), adipic acid (6 carbons), pimelic acid (7 carbons), suberic acid (8 carbons), and azelaic acid (nine carbons). Exemplary long chain diacids include diacids having more than 10, more than 15, more than 20, and more than 25 carbon atoms.

The unsaturated fatty acid-functionalized poly(glycerol diacid) material may be in the form of a scaffold, particularly a porous scaffold. Illustrative scaffold fabrication techniques include solvent casting, particulate leaching, gas foaming, phase separation, electrospinning, porogen leaching, fiber mesh, fiber bonding, self-assembly, rapid prototyping, melt molding, membrane lamination and freeze drying. One such technique is called salt leaching where salt crystals such as NaCl (common table salt) are put into a mold and polymer is poured over the salt, penetrating into all the small spaces left between the salt crystals. The polymer is hardened and then the salt is removed by dissolving it in a solvent (such as water or alcohol) which washes/leaches the salt out. Upon removal of the salt crystals all that remains is the hardened polymer with open holes/pores where the salt once was. In certain embodiments, the unsaturated fatty acid-functionalized poly(glycerol diacid) polymer may be cured in the mold in a vacuum oven at 120° C. and 100 mTorr.

One skilled in the art will recognize that other salts besides NaCl may also be employed. A porous scaffold was obtained after salt leaching in deionized water. Alternative porogens include azodicarboimide, which decomposes into nitrogen, carbon dioxide, and ammonia upon heating, and other porogens known to those skilled in the art. The primary requirements for ionic porogens are solubility in water and non-interference with polymerization.

The generated fibrous scaffolds are biodegradable and suturable.

The unsaturated fatty acid-functionalized poly(glycerol diacid) material can be electrospun into a film. In certain embodiments, the unsaturated fatty acid-functionalized poly(glycerol diacid) has a fibrous form. The unsaturated fatty acid-functionalized poly(glycerol diacid) can be used, for example, in tissue engineering such as for wound healing, vascular graft, bone regeneration and soft tissue regeneration. Tissue regeneration in situ may be accomplished by controlled degradation of the unsaturated fatty acid-functionalized poly(glycerol diacid) thus providing controlled release of the fatty acid (e.g., linoleic acid that enhances cell growth).

In certain embodiments, the unsaturated fatty acid-functionalized poly(glycerol diacid) is an elastomeric thermoset polymer.

### Applications

The elasticity of the unsaturated fatty acid-functionalized poly(glycerol diacid) material disclosed herein recommends it for use in regenerating a variety of tissues. The material may be used to tissue engineer, for example, epithelial, connective, nerve, muscle, organ, and other tissues. Exemplary tissues that can benefit from the materials of the invention include artery, ligament, skin, tendon, kidney, nerve, liver, pancreas, bladder, and other tissues. The material may also be used as the template for mineralization and formation of bone. The elastomeric material is especially recommended for regenerating tissues that are subject to repeated tensile, hydrostatic, or other stresses, such as lung, blood vessels, heart valve, bladder, cartilage and muscle.

For example, the unsaturated fatty acid-functionalized poly(glycerol diacid) material may be used to tissue engineer or regenerate a portion of a patient's bladder. In one embodiment, smooth muscle cells and urothelial cells are seeded onto the material. The cells may be allowed to proliferate before the implant is placed into a patient. To replace or regenerate cartilage, chondrocytes are seeded onto bio-rubber, which can withstand the cyclic shear and compressive forces cartilage is subjected to as joints bend.

The unsaturated fatty acid-functionalized poly(glycerol diacid) material may also be used to produce prosthetic heart valves. Heart valves are very flexible and are subjected to cyclic deformation as the heart beats. The body repairs tears in heart valve through normal physiologic mechanisms and thus can regenerate heart valves made of biodegradable materials. A heart valve seeded with smooth muscle cells and endothelial cells will be remodeled in the body to produce a new, non-synthetic heart valve. In some embodiments, it may be desirable to add fibroblasts as well. In a preferred embodiment, the regeneration occurs over a period of 3 months. The degradation rate of the polymer is easily controlled by modifying the cross-link density and/or by modifying the hydroxyl groups with hydrophobic groups.

The shape of the unsaturated fatty acid-functionalized poly(glycerol diacid) material may also be manipulated for specific tissue engineering applications. Exemplary shapes include particles, tubes, spheres, strands, coiled strands, films, sheets, fibers, meshes, and others. In one exemplary embodiment, microfabrication may be used to form capillary networks from the material. A silicon wafer is processed using standard microfabrication techniques to produce a capillary network having a desired pattern. The network is coated with a sacrificial layer, for example, sucrose. The prepolymer is cast over the sacrificial layer and cured according to the method described above. Water is used to dissolve the sacrificial layer and release the polymerized bio-rubber, which will have a relief pattern of the capillary networks that had been formed in the silicon wafer. In one embodiment, the channels in the bio-rubber are 7 μm across and 5 μm deep. One skilled in the art will realize, that while the size limit for the channels is dictated by the resolution of the microfabrication technique, biological applications may benefit from channel sizes on the order of 5 to 10's or 100's of microns or larger. The capillary networks may be closed by covering them with a flat sheet of bio-rubber and curing it.

These shapes may be exploited to engineer a wider variety of tissues. For example, the polymer may be fabricated into a tube to facilitate nerve regeneration. The damaged nerve is fed into the end of the tube, which guides the migration of axons across the wound site. Alternatively, the material may be used to fabricate the tissue structures of liver. For example, it may be formed into a network of tubes that mimic a blood vessel and capillary network which may be connected to a nutrient supply to carry nutrients to the developing tissue. Cells may be recruited to the network of tubes in vivo, or it may be seeded with blood vessel cells. Around this network of tubes, the unsaturated fatty acid-functionalized poly(glycerol diacid) material may be formed into networks imitating the arrangements of extracellular matrix in liver tissue and seeded with hepatocytes. Similarly, the material may be fabricated into a fibrous network, seeded with islet cells, and used to tissue engineer pancreas. The material may also be seeded with a variety of other cells, for example, tenocytes, fibroblasts, ligament cells, endothelial cells, epithelial cells, muscle cells, nerve cells, kidney cells, bladder cells, intestinal cells, chondrocytes, bone-forming cells, stem cells such as human embryonic stem cells or mesenchymal stem cells, and others.

Other medical applications may also benefit from the elasticity of the polymer of the invention. For example, after abdominal surgery, the intestines and other abdominal organs tend to adhere to one another and to the abdominal wall. It is thought that this adhesion results from post-surgical inflammation, however, anti-inflammatory drugs delivered directly to the abdominal region dissipate quickly. The material may be used to deliver anti-inflammatory drugs to the abdominal region. Because the material is soft and flexible, it may be implanted between the abdominal wall and internal organs, for example, by attaching it to the abdominal wall, without cutting internal organs, which would lead to infection. The anti-inflammatory drug can be released from the material over a period of months.

In another embodiment, the unsaturated fatty acid-functionalized poly(glycerol diacid) material may be used to coat a metallic stent. Because the material is flexible, it will expand with the stent without ripping, while the stiffness of the metal stent will prevent the bio-rubber from elastically assuming its previous shape. The material may release heparin or other anti-coagulants or anti-inflammatory agents to prevent the formation of clots or scar tissue, which could close off the blood vessel or throw off a thrombus that could cause circulatory problems, including stroke, elsewhere in the body.

The unsaturated fatty acid-functionalized poly(glycerol diacid) polymer may be seeded with cells, for example, connective tissue cells, organ cells, muscle cells, nerve cells, or some combination of these. In another embodiment, the polymer may further comprise a second polymer as a mixture or adduct. The second polymer may be biodegradable or non-biodegradable, and it may be biocompatible.

In particular embodiments, the disclosed unsaturated fatty acid-functionalized poly(glycerol diacid) materials and methods can be utilized to form highly porous matrices which create 3-dimensional scaffolds for cell ingrowth from the host or cell seeding for tissue engineered organ approaches. In some examples, the disclosed method is utilized to generate a vascular graft with a shaped based upon the shape of the structure, such as a blood vessel, the resulting vascular graft is replacing. In some examples, a unsaturated fatty acid-functionalized poly(glycerol diacid) tube is formed. The generated grafts are suturable.

In some examples, the fabricated scaffold or graft comprises pores of about 1 μm to about 500 μm, from about 10 μm to about 300 μm, about 20 μm to about 300 μm, about 1 μm to about 10 μm, about 3 μm to about 7 μm, such as 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, or 500 μm. In some examples, pores are about 20 μm to about 30 μm, including about 20 μm, 21 μm, 22 μm, 23 μm, 24 μm, 25 μm, 26 μm, 27 μm, 28 μm, 29 μm, and 30 μm. In some examples, the scaffold or graft is fabricated to include uniformly distributed pores. In some examples, the scaffold or graft is fabricated to include non-uniformly distributed pores. In some examples, the scaffold or graft is fabricated to not include any pores.

In some examples, the scaffold or graft is fabricated to include at least 75% pore interconnectivity, such as about 80% to about 90%, about 90% to about 98%, including 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% interconnectivity.

The various dimensions of a disclosed scaffold or vascular graft may vary according to the desired use. In some examples, the method of fabrication is performed to generate a vascular graft with an inner diameter which matches that of the host vessel to be replaced. In some examples, the inner diameter is between about 1 mm to 5 mm In some examples, a disclosed vascular graft has an inner diameter of between about 700 μm to about 5000 μm, such as about 710 μm to about 4000 μm, such as about 720 μm to about 3000 μm, such as about 1000 μm to about 5000 μm, including 710 μm, 711 μm, 712 μm, 713 μm, 714 μm, 715 μm, 716 μm, 717 μm, 718 μm, 719 μm, 720 μm, 721 μm, 722 μm, 723 μm, 724 μm, 725 μm, 726 μm, 727 μm, 728 μm, 729 μm, 730 μm, 731 μm, 732 μm, 733 μm, 734 μm, 735 μm, 736 μm, 737 μm, 738 μm, 739 μm, 740 μm, 741 μm, 742 μm, 743 μm, 744 μm, 745 μm, 746 μm, 747 μm, 748 μm, 749 μm, 750 μm, 800 μm, 850 μm, 900 μm, 950 μm, 1000 μm, 2000 μm, 3000 μm, 4000 μm or 5000 μm. In some examples, the inner diameter of a disclosed vascular graft is fabricated to be about 720 μm. In some examples, the inner diameter of a disclosed vascular graft is fabricated to be about 1000 μm. In some examples, the inner diameter of a disclosed vascular graft is fabricated to be about 1200 μm. In some examples, the inner diameter of a disclosed vascular graft is fabricated to be about 2000 μm. In some examples, the inner diameter of a disclosed vascular graft is fabricated to be about 3000 μm.

In some examples, the method of fabrication is performed to generate a vascular graft with a wall thickness which matches that of the host vessel to be replaced. However, it is contemplated the graft wall can be fabricated with a thicker or thinner wall than that which is being replaced, if desired. In some examples, a disclosed vascular graft is fabricated to have a wall thickness between about 100 μm and about 500 μm, such as about 150 μm and about 450 μm, including about 200 μm and about 400 μm, such as about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, or 500 μm. In some examples, a disclosed vascular graft is fabricated to have a wall thickness between about 270 μm and about 300 μm, such as about 285 μm and about 295 μm, including 270 μm, 271 μm, 272 μm, 273 μm, 274 μm, 275 μm, 276 μm, 277 μm, 278 μm, 279 μm, 280 μm, 281 μm, 282 μm, 283 μm, 284 μm, 285 μm, 286 μm, 287 μm, 288 μm, 289 μm, 290 μm, 291 μm, 292 μm, 293 μm, 294 μm, 295 μm, 296 μm, 297 μm, 298 μm, 299 μm, or 300 μm. In some examples, the wall thickness is about 290 μm.

In some examples, the method of fabrication are performed to generate a scaffold or vascular graft that at least 50%, such as about 55% to about 70%, about 80% to about 90%, about 90% to about 98%, including 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% of such vascular graft degrades within one year, such as within 1 to 10 months, including within 1 month, 2 months, 3 months, 4 months, 5 months, 6 months, 7 months, 8 months, 9 months, 10 months, 11 months, or 12 months of implantation. Degradation of the graft permits controlled and/or sustained release of the fatty acid (e.g., linoleic acid).

In some examples, the method of fabrication includes generating a cell-free scaffold or graft, such as a cell-free vascular graft, in which the graft does not include any living cells, such as smooth muscle cells, endothelial cells, stem cells, or progenitor cells.

In some examples, the disclosed methods of fabrication include impregnating or coating a surface of a generated fiber, scaffold or graft with one or more, such as two, three, four, five etc. suitable pharmaceutical agents. It is contemplated that suitable pharmaceutical agents can be organic or inorganic and may be in a solid, semisolid, liquid, or gas phase. Molecules may be present in combinations or mixtures with other molecules, and may be in solution, suspension, or any other form. Examples of classes of molecules that may be used include human or veterinary therapeutics, cosmetics, nutraceuticals, agriculturals such as herbicides, pesticides and fertilizers, vitamins, salts, electrolytes, amino acids, peptides, polypeptides, proteins, carbohydrates, lipids, nucleic acids, glycoproteins, lipoproteins, glycolipids, glycosaminoglycans, proteoglycans, growth factors, hormones, neurotransmitters, pheromones, chalones, prostaglandins, immunoglobulins, monokines and other cytokines, humectants, metals, gases, minerals, plasticizers, ions, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules.

Pharmaceutical agents include any therapeutic molecule including, without limitation, any pharmaceutical substance or drug. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfiram and disulfiram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor. All substances listed by the U.S. Pharmacopeia are also included within the substances of the present disclosure.

In some examples, the inner luminal surface of a biodegradable scaffold is coated partially or completely with a thromboresistant agent, such as heparin and/or other compounds known to one of skill in the art to have similar anti-coagulant properties as heparin, to prevent, inhibit or reduce clotting within the inner lumen of the vascular graft.

In certain embodiments, a salt leaching technique may be used to make tubes or disk to give adapted shape for the use. With salt leaching technique, highly porous scaffolds, with a range of 74-77% of porosity may obtained depending on the salt crystal size (varying 25 -150 um) and packing skills.

The polymers disclosed herein may be electrospun to form scaffolds of any desired shape, such as sheets, tubes, meshes, pseudo 3-dimensional constructs. It is contemplated that the constructs may be of high porosity, low porosity, a combination of different porosity. In some examples, the methods are utilized to form vascularized (micro-channeled) fibrous sheets, random meshes, aligned sheets, cylindrical tubes, or pseudo 3-dimensional constructs, such as shapes to mimic organs. In some examples, complex shapes such as those mimicking organs are formed by the disclosed methods. The disclosed methods of electrospinning with a sacrificial template can be used to create highly porous scaffolds to mimic ECM. These structures are especially useful for applications in soft and elastomeric tissues. It is contemplated that the disclosed methods can be used to generate constructs/scaffolds used to guide host tissue remodeling in many different tissues, including any tissue that has progenitor cells. The biodegradable scaffold can be used to facilitate tissue regeneration in vivo by providing a structural frame for which tissue regeneration can occur. In some examples, the polymer is electrospun such that it allows and facilitates the infiltration of host cells including progenitor cells. In some examples, the composition is such that it allows and facilitates host remodeling of the polymer, so that eventually the polymeric structure is replaced by the desirable host tissue. It is contemplated that the methods of fabrication disclosed herein can be modified as desired by one of ordinary skill in the art to fabricate a graft with the appropriate dimensions and features depending upon tissue which is to be replaced.

In some particular examples, the generated tissue constructs are for the replacement and/or repair of damaged native tissues. For example, the disclosed constructs are contemplated to be implantable for tensile load bearing applications, such as being formed into tubular networks with a finite number of inlets and outlets. These structures can be either seeded with cells or implanted directly and relying on the host to serve as cell source and “bioincubator”. These structures can be implanted as artificial organs and the inlets and outlets will be connected to host vasculature. Porous morphology can be varied. It is contemplated that the disclosed methods can be used to generate stronger constructs, such as constructs to be directly implanted in subjects into load bearing environments without additional mechanical support. Uses range from vascularized sheets for hernia repair, prolapse, and wound dressings, to complex tubes for blood vessel, nerve and trachea repair. Additionally, aligned random transition spinning may be useful for ligament-bone interfaces.

In some examples, the vasculature itself maybe valuable without parenchymal cells. For example in treating ischemic diseases. The microvascular mimetics can be connected directly to a host vessel and quickly perfuse an ischemic area of the body.

Disclosed herein are scaffolds, such as tissue engineering scaffolds, including for the replacement and/or repair of damaged native tissues. Although the present disclosure illustrates in detail the use of a disclosed scaffold within a vascular graft, it is contemplated that a disclosed scaffold can be utilized for additional in situ tissue engineering applications, including, but not limited to bone, intestine, liver, lung, or any tissue with sufficient progenitor/stem cells. In certain embodiments, the disclosed scaffold could be used in a heart valve. In some examples, a scaffold is biodegradable and/or biocompatible and includes a biodegradable core, such as a biodegradable polyester tubular core for a vascular graft. In certain embodiments, the polymer disclosed herein may be used as a tubular core and a biodegradable polyester (e.g., poly(caprolactone)(PCL)) outer sheath surrounds the core thus forming a vascular graft. Illustrative scaffolds and outer sheaths are described, for example, in PCT Appl. No. PCT/US2016/037790, which is incorporated herein by reference. Certain embodiments are described in the following numbered clauses:

1. A polymer comprising repeating units having a structure of:

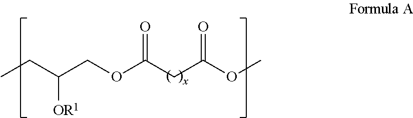

wherein x is 2 to 25; and

each R¹ is the same or different and is a moiety derived from an unsaturated fatty acid, a dicarboxylic acid, or a dicarboxylic acid-terminated polymer.

2. A polymer comprising an unsaturated fatty acid-functionalized poly(glycerol diacid).

3. The polymer of clause 1, wherein at least one R¹ is derived from a polyunsaturated omega-6 fatty acid or a polyunsaturated omega-3 fatty acid.

4. The polymer of clause 3, wherein the unsaturated fatty acid is a polyunsaturated omega-6 fatty acid or a polyunsaturated omega-3 fatty acid.

5. The polymer of any one of clauses 1 to 4, wherein the polyunsaturated omega-6 fatty acid is linoleic acid (LA), gamma linolenic acid (GLA), arachidonic acid (AA), epoxyeicosatrienoic acid, or conjugated linoleic acid (CLA).

6. The polymer of any one of clauses 1 to 4, wherein the polyunsaturated omega-3 fatty acid is docohexaenoic acid.

7. The polymer of clause 2, wherein the diacid is sebacic acid.

8. The polymer of clause 2 or 7, wherein the unsaturated fatty acid is linoleic acid.

9. The polymer of clause 2, wherein the polymer is poly(glycerol sebacate linoleate).

10. The polymer of clause 1, wherein at least one R¹ is linoleate.

11. The polymer of clause 1 or 10, wherein x is 8.

12. The polymer of any one of clauses 1, 10, or 11, wherein the polymer includes a plurality of crosslinked units of formula A crosslinked at the R¹ position and at least one unit of formula A wherein R¹ is a moiety derived from an unsaturated fatty acid.

13. A prepolymer comprising:

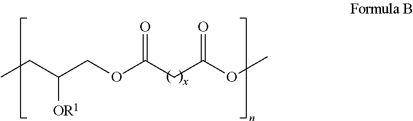

wherein n is 2 to 12;

x is 2 to 25; and

each R¹ is the same or different and is a moiety derived from an unsaturated fatty acid, a dicarboxylic acid, or a dicarboxylic acid-terminated polymer.

14. The prepolymer of clause 13, wherein R¹ is derived from a polyunsaturated omega-6 fatty acid or a polyunsaturated omega-3 fatty acid.

15. A material comprising a porous scaffold comprising the polymer of any one of clauses 1 to 12.

16. A vascular graft comprising the scaffold of clause 15.

17. A vascular graft comprising the polymer of any one of clauses 1 to 12.

18. A method comprising reacting together a poly(glycerol diacid) and an unsaturated fatty acid.

19. The method of clause 18, wherein the poly(glycerol diacid) is a poly(glycerol sebacate) prepolymer.

20. The method of clause 18 or 19, wherein the unsaturated fatty acid is a polyunsaturated omega-6 fatty acid or a polyunsaturated omega-3 fatty acid.

21. The method of any one of clauses 18 to 20, wherein the reaction is effected under conditions sufficient for forming a porous scaffold.

### EXAMPLES

### Synthesis of PGS Prepolymer

PGS was synthesized by polycondensation of 0.1 mole each of glycerol (Aldrich, Milwaukee, Wis.) and sebacic acid (Aldrich) at 120.degree. C. under argon for 24 h before the pressure was reduced from 1 Torr to 40 mTorr over 5 h. The reaction mixture was kept at 40 mTorr and 120.degree. C. for 48 h. Polycondensation of glycerol and sebacic acid yields a transparent, almost colorless elastomer that does not swell or dissolve in water.

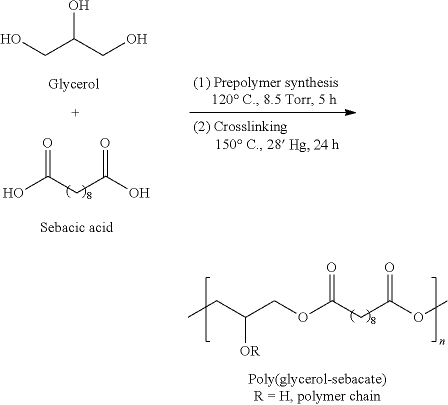

### Synthesis of PGSL Prepolymer Followed by Curing

Linoleic acid (3.5 mmol, 0.98 g) was added dropwise to the solution of N,N′-Dicyclohexylcarbodiimide (DCC) (3.85 mmol, 0.79 g, 1.1 equiv.) and 4-Dimethylaminopyridine (DMAP) (0.35 mmol, 42 mg) in dry THF (15 ml), at 0° C. under inert condition. After 2 h of reaction at room temperature, 1 g of PGS prepolymer in dry THF (25 mL) solution was added to the reaction mixture at 0° C., then the reaction occurred for 3 h at r.t. The crude product was purified by 3 times of centrifugation at 3000 rpm for 10 min. Yellowish supernatant was collected and dried by solvent evaporation.

600 mg of the resulting PGSL prepolymer was dissolved in THF (3 mL) loaded to the salt template with sieve range=75-150 nm. After complete solvent evaporation, PGSL was cured in an oven at 140° C. for 24 h under high vacuum atmosphere (100 mTorr). After completion of curing process, the salt was washed out in a DI water bath overnight. The final PGSL form was obtained by lyophilizing for 24 h.

### One-Step Synthesis of PGSL

LA (350 mg, 1.3 mmol) was completely dissolved in 3 mL of THF with PGS prepolymer (30 mg). The mixture was loaded into a salt template with sieve diameter=75-150 nm. The curing process was first occurred at 140° C. for 4 h without vacuum, then continued to cured for 20 more hours under high vacuum (100 mTorr). After completion of curing process the salt was removed in the same manner as described above.

### PGSL Film Fabrication

PGS prepolymer (400 mg) and LA (420 mg) were dissolved in 1 mL of THF. The solution was transferred and dried in a hyaluronic acid pre-treated Petri dish. Hyaluronic acid was used as the detaching agent from the glass after completion of Polymer curing. The dried PGS and LA mixture was cured in the oven at 140° C. for 24 h. The final yellowish film was obtained by removing hyaluronic acid in a DI water bath.

### PGSL Film Wettability Test

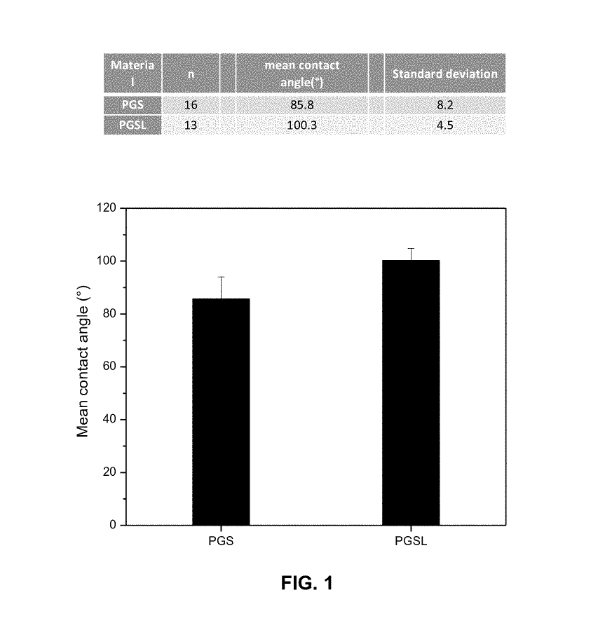

PGS and PGSL films were both prepared by thermal curing process in an oven at 150° C. for 24 h with high vacuum (100 mTorr). The contact angle of DI water on the PGS and PGSL films was detected by FireWire camera with telecentric optics and 50 mm focus and 40 mm extension tube, its value was calculated automatically by Theta Lite program using Young-Laplace equation for both sides of contact angles of drop. The mean values contact angle θ (°) of films are reported in the table shown in FIG. 1.

The wettability was tested with a water drop on the clean films. The mean contact angle for PGSL was 100.3° versus 85.8° for PGS. Resulting contact angle on PGSL film is slightly higher than PGS, as expected, due to the hydrophobic aspect of PGSL.

### DSC Test

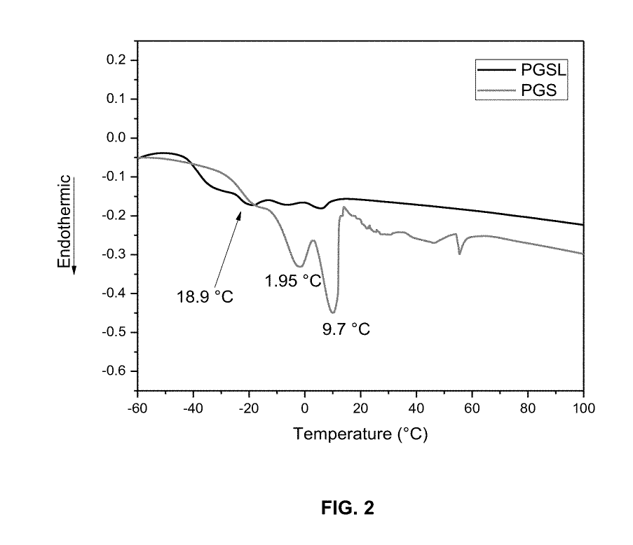

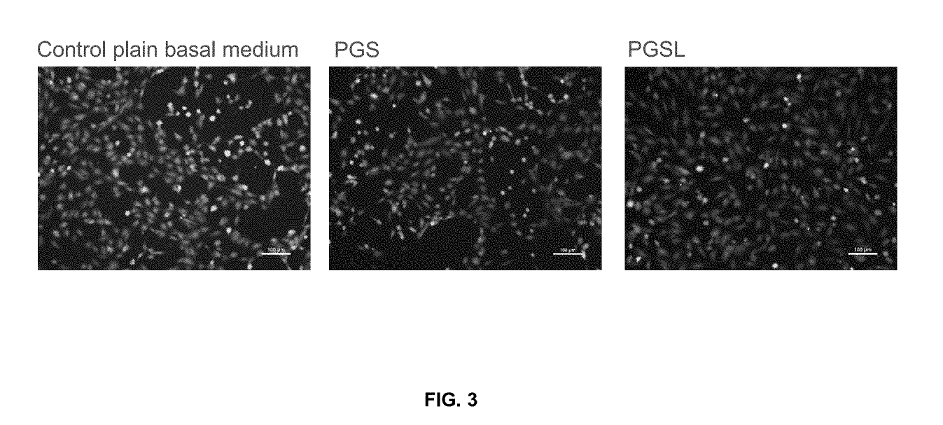

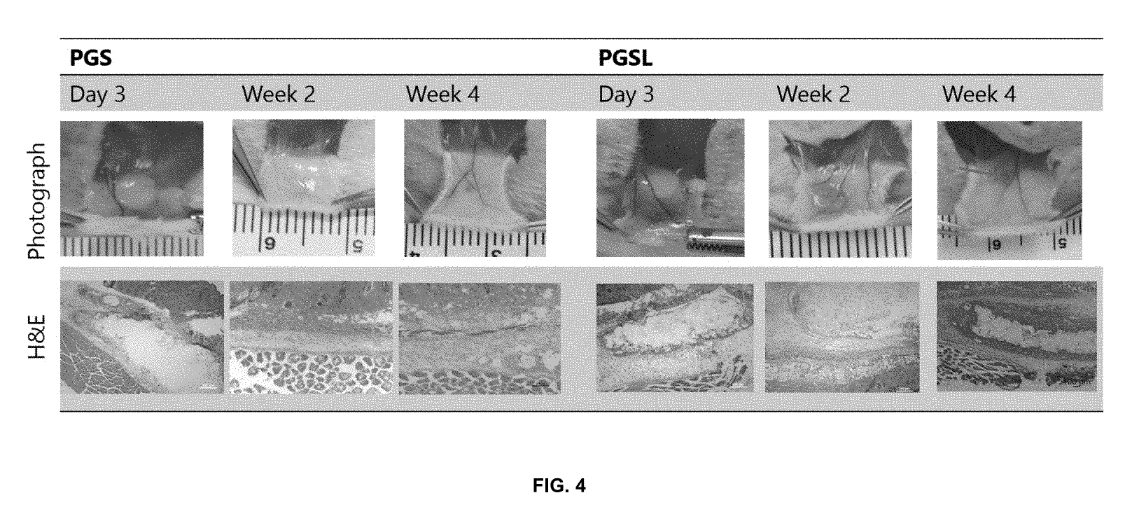

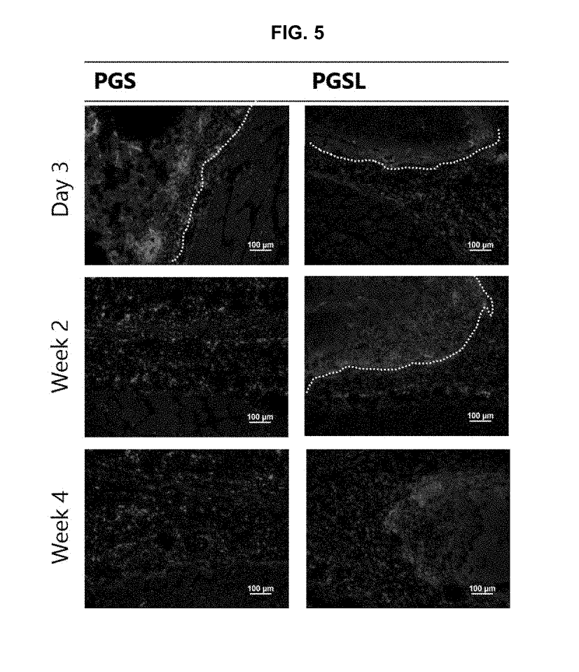

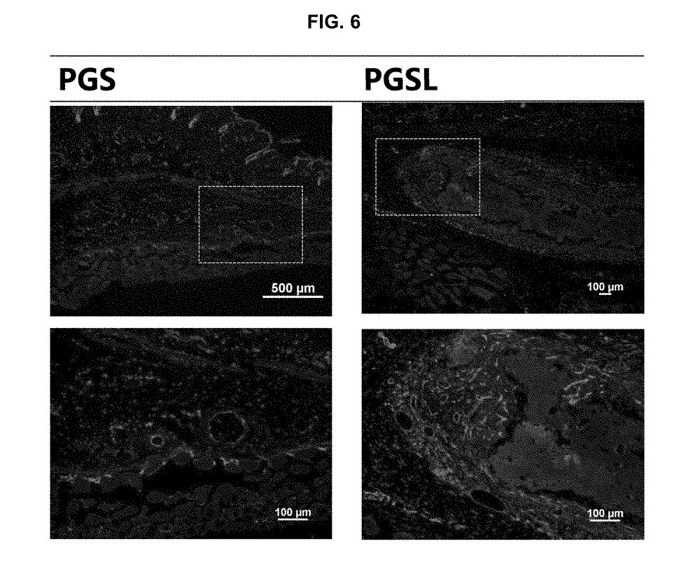

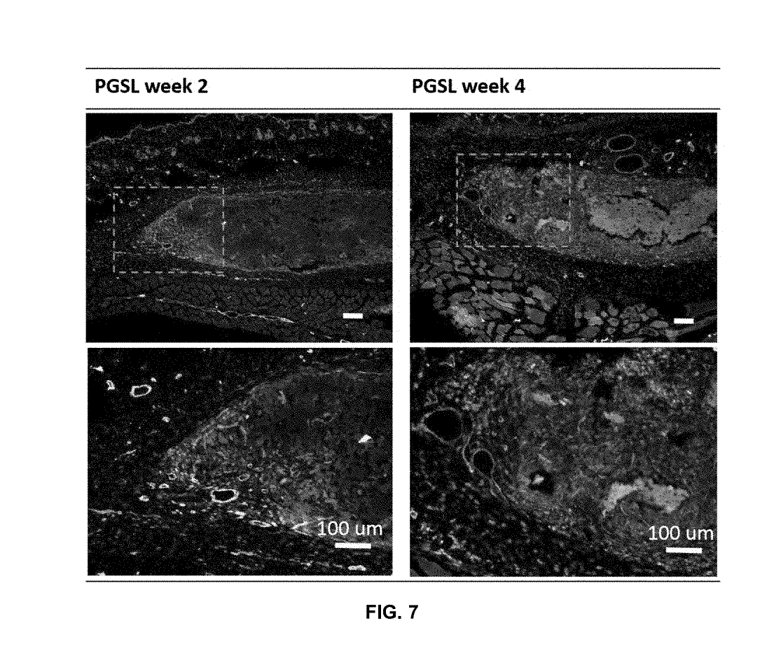

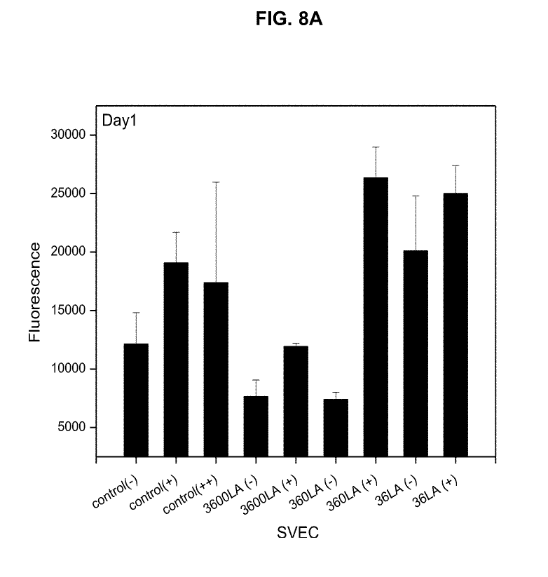

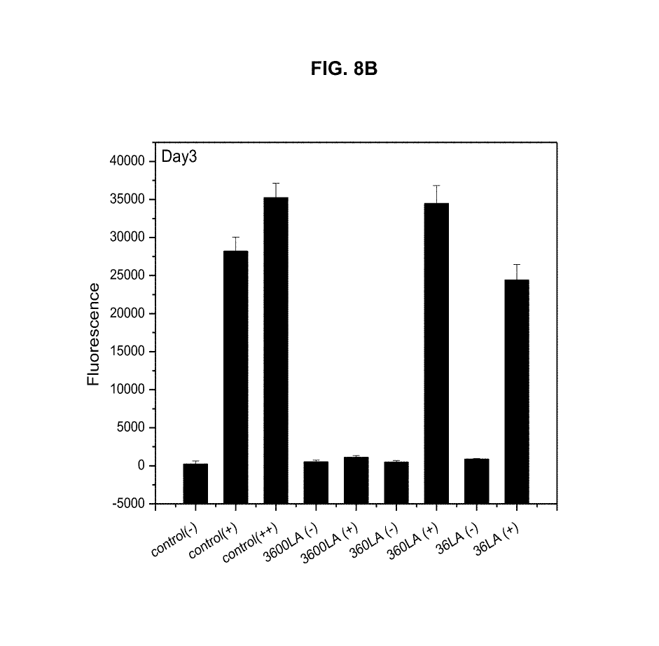

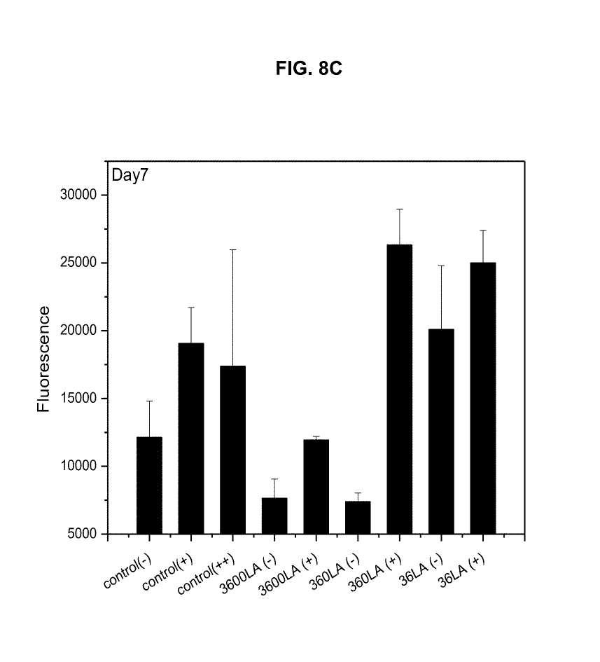

Differential scanning calorimetry (DSC) traces of samples of PGSL and PGS film recorded under nitrogen. The samples were first heated from 25° C. to 150° C., cooled from 150° C. to −75° C., and heated again from −75° C. to 250° C. All heating and cooling rates were 10° C./min. The results are shown in FIG. 2.

### Cell Viability Assay

The cell media used was the EGM-2 Bullet Kit (CC-3162) from Lonza. It consists of 500 mL of basal media, antibiotics GA-1000 (Gentamicin, Amphotecerin-B), FBS (Fetal Bovine Serum) 10 mL, and a kit of growth factors (VEGF, hFGF-B, R3-IGF-1, Ascorbic Acid, Heparin).
In vitro viability assay:Live/dead assay
In vitro cytotoxicity(biocompatibility)/proliferation assay: CellTiterBlue® assay
This in vitro test was to find the optimal linoleic acid content in the material. Cells were cultured in different concentration of LA. This test was allowed to determine the cytotoxic amount of LA and its proliferation ability at a time.
After incubating 1 000 vascular endothelial cells of sheep (SVEC) that were seeded in each of 96-well plates at 37° C. for 24 h to attach, the EGM-2 basal medium is changed to different culture media:
Control(−) Plain EGM-2 basal medium with antibiotics,
Control+) EGM-2 with antibiotics and Serum,
Control(++) EGM-2 with Serum and Growth Factor,
3600LA (−) 0.0001 w/v % LA (3.6 μM) in control (−)medium
3600LA (+) 0.0001 w/v % of LA in control (+)medium
360LA (−) 0.00001 w/v % of LA (360 nM) in control (−)medium
360LA (+) 0.00001 w/v % of LA in control (+)medium
36LA (−) 0.000001 w/v % of LA (36nM) in control (−)medium
36LA (+) 0.000001 w/v % of LA in control (+)medium

The fluorescence of live cells were measured at three time points:1, 3, 5 days. At each time point, cells were treated with 20 μl of CellTiter-Blue (resazurin) solution and incubated for an additional 4 h at 37° C. Fluorescence was recorded triple times for each well at 560 nm/590 nm excitation/emission.

### Cytotoxicity of PGSL Material was Tested by Live/Dead Assay

The material extract cytotoxicity testing was performed according to ISO 10993-5 and the instructions of the LIVE/DEAD assay kit manufacturers. 20 mg of each PGS and PGSL polymers is incubated in culture medium at 37° C. for 24 hours. The supernatant of the medium solution is ready to be use for the cell culture.
5,000 HUVECs were seeded in into a 96-well plate. After letting the cells to attach to the well for 24 h, the culture media was exchanged by extraction media. Plain culture media that had incubated alongside the extract was given to control wells. After incubation of cells at 37° C. for 24 h, Cells were washed with DPBS, and a 100 ml solution of DPBS with 10 mmol calcein AM and 10 mmol ethidium homodimer-1 (LIVE/DEAD assay kit) was applied. Fluorescent images in the FITC (calcein) and TRITC (ethidium) channels were captured after at least 30 min of incubation at r.t. The numbers of live and dead cells were counted using the Nikon Ti Eclipse image analysis software in three randomly chosen 100× fields in each of three wells per treatment group.

### Subcutaneous Implantation of PGSL in Mice

PGS and PGSL forms that were cut in 3 mm Ø circle shape pad were sterilized in autoclave 20 min up to 140° C., then washed consecutively in 75% of EtOH, 50% EtOH, 25% EtOH and sterile saline solution. Under sterile conditions, material was implanted subcutaneously in the right and left back of 8-week-old male mice weighing 22-25 g after brief anesthestization with 2-5% isoflurane Animals were cared for in compliance with a protocol approved by the Committee on Animal Care of the University of Pittsburgh, following National Institutes of Health guidelines for the care and use of laboratory animals Mice were euthanized and samples collected at 3, 5, 7, 15, and 28 days after implantation. Tissues were fixed in 10% neutral buffered formalin for 15 min before histological analysis. Tissues were soaked in 30% sucrose and embedded in O.C.T. Six-micrometer-thick sections were cut along each sample. Sections were stained using conventional histology protocols with hematoxylin and eosin (H&E). EC migration and inflammatory cells presence were determined by immunofluorescence staining using CD31 (EC marker),a-sma (mural cells of blood vessel marker), CD68 (macrophage, monocyte marker) and DAPI was used as counterstaining.

### Degradation Rate

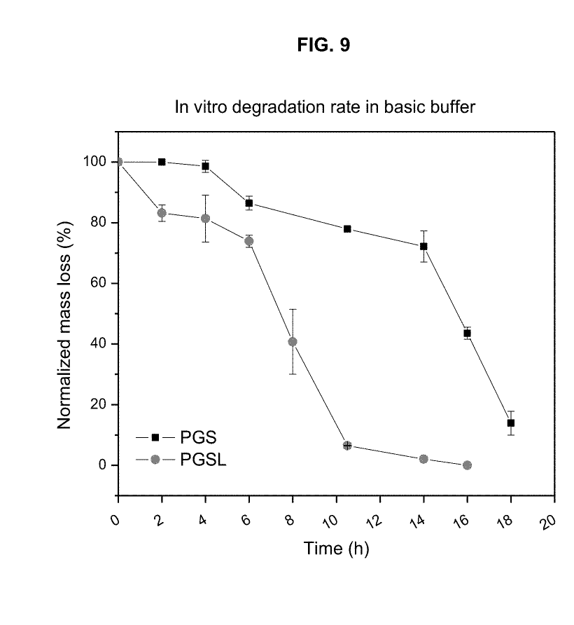

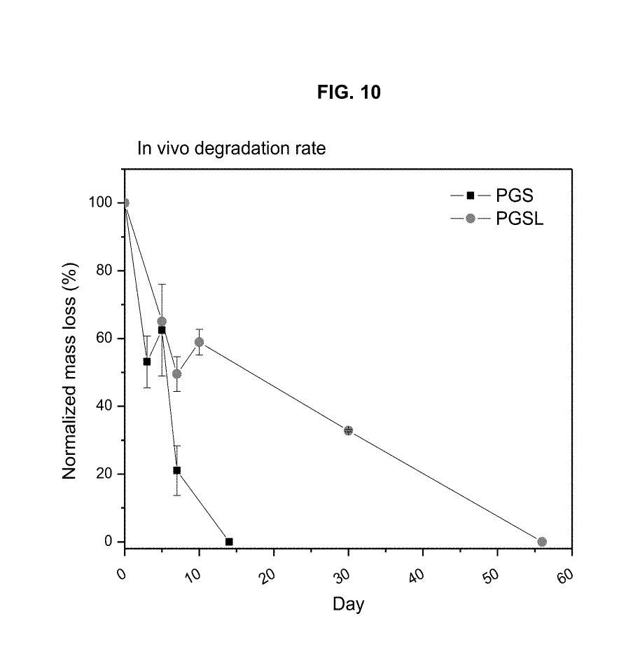

The degradation rate was studied in vitro and in vivo (see results shown in FIGS. 9 and 10). This study shows the controlled release of the bioactive fatty acids. The mass loss of simple PGS and modified PGSL containing 30 molecular weight % of linoleic acid were recorded in basic buffer (pH=11.5, in vitro) and in physiological condition (in vivo, PGSL scaffold was implanted subcutaneously in mice). Basic buffer induced hydrolysis of ester bonds in PGSL. This chemical decomposition of PGSL occurred faster than PGS scaffold due to its slightly lower crosslinking density compared to the PGS. In in vivo condition, PGS and PGSL were degraded mostly by enzymatic reaction, thus the hydrolysis is generally much slower in vivo than in vitro, and the complete degradation in vivo of PGSL took about 7-8 weeks. This result demonstrated that PGSL degrades 4 times slower than conventional PGS materials. This opposite result between in vitro and in vivo is due to the higher hydrophobicity of PGSL in presence of fatty acid (linoleic acid) on the polymeric chains.

### Bioactivity

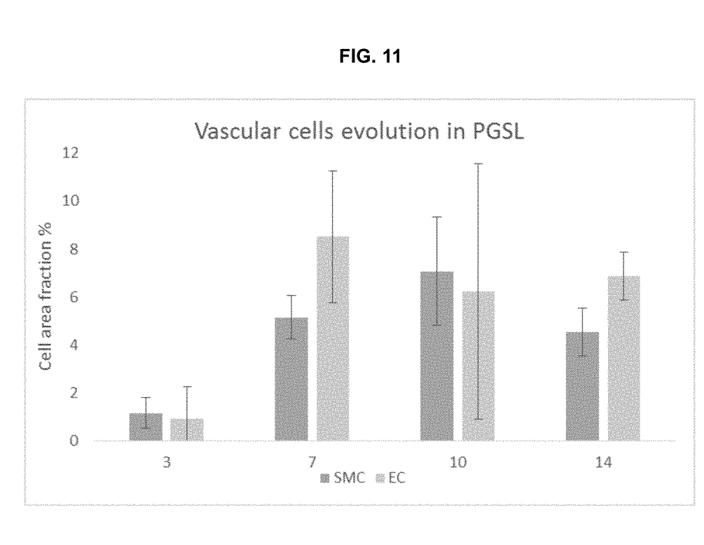

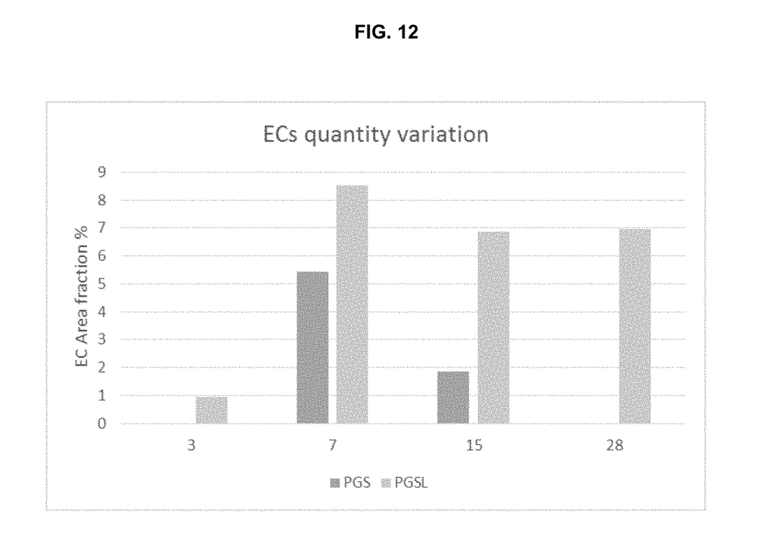

To observe the bioactivity of reported invention, PGSL, in angiogenesis in vivo, the vascular endothelial cells (ECs) and vascular smooth muscle cells (SMCs) quantity variations were determined by ECs and SMCs staining of the tissue in where the PGSL scaffold was implanted (mice) (see the results in FIGS. 11 and 12). This analysis was arranged using histochemistry with marker CD31(EC marker) and alpha smooth muscle actin (SMC marker). The evolution of ECs and SMCs infiltration into the PGSL scaffold were monitored at different time points (day 3, 7, 10, and 14).

ECs variation in PGSL over the time was compared to the EC variation in PGS. Infiltration of vascular cells at early time point was observed for our invention, PGSL samples. Moreover the quantity of infiltrated vascular cells in vivo were higher than existent material PGS.

In view of the many possible embodiments to which the principles of the disclosed invention may be applied, it should be recognized that the illustrated embodiments are only preferred examples of the invention and should not be taken as limiting the scope of the invention.

What is claimed is:

1. A polymer comprising repeating units having a structure of: 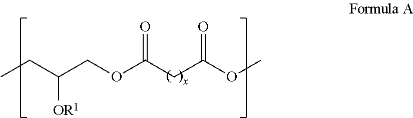
wherein x is 2 to 25; and
each R¹ is the same or different and is a moiety derived from an unsaturated fatty acid, a dicarboxylic acid, or a dicarboxylic acid-terminated polymer.

2. A polymer comprising an unsaturated fatty acid-functionalized poly(glycerol diacid).

3. The polymer of claim 1, wherein at least one R¹ is derived from a polyunsaturated omega-6 fatty acid or a polyunsaturated omega-3 fatty acid.

4. The polymer of claim 2, wherein the unsaturated fatty acid is a polyunsaturated omega-6 fatty acid or a polyunsaturated omega-3 fatty acid.

5. The polymer of claim 3, wherein the polyunsaturated omega-6 fatty acid is linoleic acid (LA), gamma linolenic acid (GLA), arachidonic acid (AA), epoxyeicosatrienoic acid, or conjugated linoleic acid (CLA).

6. The polymer of claim 4, wherein the polyunsaturated omega-6 fatty acid is linoleic acid (LA), gamma linolenic acid (GLA), arachidonic acid (AA), epoxyeicosatrienoic acid, or conjugated linoleic acid (CLA).

7. The polymer of claim 3, wherein the polyunsaturated omega-3 fatty acid is docohexaenoic acid.

8. The polymer of claim 4, wherein the polyunsaturated omega-3 fatty acid is docohexaenoic acid.

9. The polymer of claim 2, wherein the diacid is sebacic acid.

10. The polymer of claim 2, wherein the unsaturated fatty acid is linoleic acid.

11. The polymer of claim 2, wherein the polymer is poly(glycerol sebacate linoleate).

12. The polymer of claim 1, wherein at least one R¹ is linoleate.

13. The polymer of claim 1, wherein x is 8.

14. The polymer of claim 1, wherein the polymer includes a plurality of crosslinked units of formula A crosslinked at the R¹ position and at least one unit of formula A wherein R¹ is a moiety derived from an unsaturated fatty acid.

15. A prepolymer comprising: 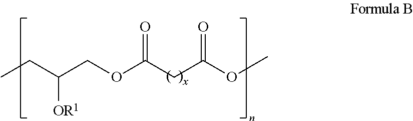
wherein n is 2 to 12;
x is 2 to 25; and
each R¹ is the same or different and is a moiety derived from an unsaturated fatty acid, a dicarboxylic acid, or a dicarboxylic acid-terminated polymer.

16. The prepolymer of claim 15, wherein R¹ is derived from a polyunsaturated omega-6 fatty acid or a polyunsaturated omega-3 fatty acid.

17. A material comprising a porous scaffold comprising the polymer of claim 1.

18. A material comprising a porous scaffold comprising the polymer of claim 2.

19. A vascular graft comprising the scaffold of claim 17.

20. A vascular graft comprising the scaffold of claim 18.

21. A vascular graft comprising the polymer of claim 1.

22. A polymer made by reacting together a poly(glycerol diacid) and an unsaturated fatty acid.

23. A method comprising reacting together a poly(glycerol diacid) and an unsaturated fatty acid.

24. The method of claim 23, wherein the poly(glycerol diacid) is a poly(glycerol sebacate) prepolymer.

25. The method of claim 23, wherein the unsaturated fatty acid is a polyunsaturated omega-6 fatty acid or a polyunsaturated omega-3 fatty acid.

26. The method of claim 23, wherein the reaction is effected under conditions sufficient for forming a porous scaffold.
